(19) United States
(12) Patent Application Publication
(10) Pub. No.: US 2007/0190571 A1
(43) Pub. Date: Aug. 16, 2007

(54) DETECTION OF AN EXOGENOUS LIGAND IN BIOLOGICAL FLUID

(76) Inventors: Barry M. Forman, Irvine, CA (US); Xiaohui Yuan, Arcadia, CA (US)

Correspondence Address: PERKINS COIE LLP, POST OFFICE BOX 1208, SEATTLE, WA, 98111-1208, US

(21) Appl. No.: 11/539,831

(22) Filed: Oct. 9, 2006

Related U.S. Application Data
(60) Provisional application No. 60/725,451, filed on Oct. 11, 2005.

(51) Int. Cl. G01N 33/53; G01N 33/92

(52) U.S. Cl. 435/7.1; 436/71

(57) ABSTRACT

Methods of and screens for detection of exogenous ligands in a biological sample without prior knowledge of the chemical structure of the ligand are disclosed. The novel screens and methods may be used to detect Illicit use of performance-enhancing steroids, which use has proliferated among a wide range of professional and amateur athletes. While these athletes were previously able to evade detection because the steroids were unknown to authorities, the present strategies can successfully detect such “designer steroids” without prior knowledge of their existence.

### CROSS-REFERENCE TO RELATED APPLICATIONS

This application claims the benefit of U.S. Provisional Patent Application No. 60/725,451, filed Oct. 11, 2005, and this application is incorporated herein by reference.

### GOVERNMENT INTEREST

The research in the present application was supported, in part, by an NIH NURSA pilot grant, Grant No. U19 DK62434. The Government has certain rights in the invention.

### BACKGROUND

Each of the references cited herein is incorporated by reference in its entirety. A complete listing of the citations is set forth at the end of the specification.

The illicit use of performance-enhancing drugs has proliferated among elite professional athletes, Olympians and even high-school level amateurs. The widespread abuse of these potentially toxic agents has aroused Congressional and public attention (1). The success of proposed legislation to increase testing and punishment of professional athletes who use illicit steroid assumes the availability of robust screens that can detect the presence of all performance-enhancing drugs. In the case of anabolic steroids, current approaches utilize sensitive assays such as gas chromatography/mass spectrometry or enzyme-linked immunosorbent assay to detect the presence of known steroids in biological samples. These assays suffer from a severe limitation because they require pre-existing knowledge of the precise compounds that are being abused. An athlete can evade detection by using an anabolic steroid that is not known to authorities (2). Indeed, an emerging clandestine industry has allowed athletes to evade detection via the use of novel “designer steroids.” Several test-evading designer steroids have been identified in the past three years including two existing compounds that were never marketed (3) and a novel chemical entity named tetrahydrogestrinone that was specifically synthesized to evade detection (4). The latter drug has been implicated in the 2004 BALCO investigation.

Anabolic steroids function by binding to the nuclear androgen receptor (“AR”) (2). A vast array of diverse synthetic ligands have been identified for AR and other nuclear receptors (5) and hundreds or even thousands of AR ligands have been described in the patent and scientific literature, although they have never been marketed. Moreover, existing high-throughput screening strategies could allow for the design and production of additional designer steroids. These test-evading steroids remain in use because the structure of the compounds is not known and thus the compounds are not identified. In these cases, it is typically an insider who reveals the existence of the compound to authorities. It is evident that novel strategies must be developed that can detect illicit steroids without prior knowledge of their chemical structure. To be useful in an anti-doping setting, these assays must also distinguish illicit androgens from their naturally occurring counterparts: testosterone (“T”) and dihydrotestosterone (“DHT”). The methods and screens described herein are useful for this purpose and may also be used for other situations in which it is advantageous to identify a steroid without knowing its precise chemical structure.

The problems identified in the prior art are not all the problems in the prior art. The foregoing examples of the related art and limitations related therewith are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

Methods of and screens for detecting exogenous ligands, such as illicit, performance-enhancing steroids, without prior knowledge of their existence and/or chemical structure, are disclosed. The methods and screens allow for the identification of athletes who seek to evade detection by using novel compounds that were previously undetectable because they are unknown to authorities. The method examines an unknown compound (i.e., the substance at issue) bound to one or more known receptors and then compares the chemical activity of the unknown substance to that of a known, endogenous substance that binds to the same receptor. If the results, which may be a peak on a chromatogram, evidence that the unknown substance does not behave in the same way and/or is chemically distinct from an endogenous substance, then the unknown substance is an exogenous substance, such as a designer steroid. Another aspect of the present methods and screens allows for detecting artificial or exogenous ligands bound to known receptors.

In addition to the exemplary aspects and embodiments described above, further aspects and embodiments will become apparent by reference to the drawings and by study of the following descriptions.

### BRIEF DESCRIPTION OF FIGURES

FIG. 1: (A) Normal human serum was incubated with control or AR-containing beads. Eluants were analyzed by gas chromatography/mass spectrometry (“GC/MS”) and the chromatograms were overlaid. The peaks corresponding to DHT (11.37 minutes) and T (11.67 minutes) were confirmed by comparing elution times and mass spectra with known standards. (B) Normal human serum containing 1 nm nandrolone was analyzed as in (A). The elution time and mass spectrum of the 11.42 minutes peak is indistinguishable from nandrolone.

### DETAILED DESCRIPTION

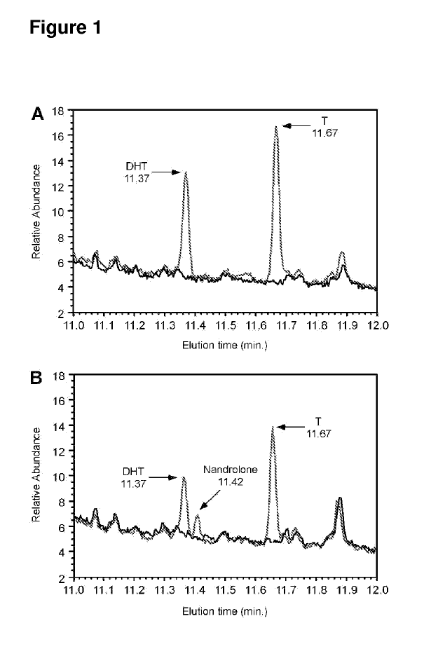

Method and screens for detecting one or more exogenous ligands in a subject are disclosed. “Exogenous ligand” means a molecule that binds to a receptor, wherein said molecule or ligand originates outside of the organism in which it is found. Generally, a biological sample from the subject is obtained first. The biological sample may be any sample that would contain the exogenous ligand if it were present in the subject. For example, potentially acceptable biological samples include blood, serum, plasma, urine, feces, bile, tears, lymphatic fluid, cerebrospinal fluid, or saliva. Next, the sample is incubated with the receptor that would bind the exogenous ligand if the exogenous ligand were present. More than one exogenous ligand may be tested for at a time. The receptor may be bound to a support medium, such as a bead, which may be agarose, sepharose, latex or magnetic, nylon membrane, matrix, microtitre well, column, nitrocellulose, polyvinylchloride, polystyrene, diazotized paper, or plastic among other suitable materials, which are known in the art. Once the biological sample has had sufficient incubation to the receptor to allow binding if corresponding ligands are present, the receptors are washed with a substance that removes the ligands that are bound to the receptors, such as ligand binding buffer. The ligand removed from the receptors may then be concentrated, purified, dried, and/or otherwise prepared. The final step is the analysis of the removed ligand, which determines whether an exogenous ligand is present in the sample in addition to any endogenous ligands that bind to the same receptor. The analysis compares the results obtained from incubating the one or more unknown ligands (exogenous or endogenous) in the sample with: 1) a known receptor attached to a support medium, 2) a receptor-free control such as a support medium, and/or 3) a control protein attached to a support medium, which is free of the exogenous ligand. The method or screen may also determine the presence of the exogenous ligand by comparing analytical results from the sample to the results of the endogenous ligand, the same exogenous ligand, and/or a different exogenous ligand. If a substance is seen in analysis of the sample, such as additional peak(s) in a CG/MS graph accompanying the peaks representing the endogenous ligands (See FIG. 1), then the biological sample contains an exogenous ligand.

There are many known methods of testing whether a ligand is present or absent, including gas chromatography/mass spectrometry, liquid chromatography/mass spectrometry, liquid chromatography multistage mass spectrometry and nuclear magnetic resonance (NMR). These methods may be used alone or in combination for additional verification. One skilled in the art will be able to determine which method or methods are most effective.

A ligand of interest may be a steroid, such as a corticosteroid, estrogen, progesterone, or androgen. For the use of detecting illicit, performance-enhancing steroids, the steroid will likely be an anabolic steroid and the receptor for that steroid, the androgen receptor. Thus, the present methods and screens can be used for maintaining the integrity of athletic competitions, both amateur and professional, by testing athletes, human or animal, for the use of illicit steroids, and banning those athletes who test positive.

A sensitive assay is established that identifies a ligand, such as an anabolic and/,or other ligand, by virtue of its ability to bind to its pharmacologic target, which is usually a receptor such as the androgen receptor (“AR”). Then, the bound ligand is analyzed by a method, alone or in combination, such as gas chromatography/mass spectrometry (“GC/MS”), liquid chromatography/mass spectrometry, liquid chromatography multistage mass spectrometry or NMR, which distinguishes the exogenous ligand from endogenous ligands. The exogenous ligand may be an illicit drug and the endogenous ligand may be natural androgens produced by the body. A significant benefit of this screen is the ability to determine the presence of exogenous ligands for any receptor, without knowing the chemical structure of the steroid. Table 1 lists steroid receptors (both standardized name and common name) and their natural ligands, all of which may be used in the present methods and screens. Of course, this method may also be used with other receptors not present in Table 1.

TABLE 1

| Official | | |
|---|---|---|
| name | Common name | Natural Ligand |
| NR0B1 | DAX1 | Unknown |
| NR0B2 | SHP | Unknown |
| NR1A1 | TRa | Thyroid hormones (T3, T4) |
| NR1A2 | TRb | Thyroid hormones (T3, T4) |
| NR1B1 | RARa | Retinoids (e.g. retinoic acid) |
| NR1B2 | RARb | Retinoids (e.g. retinoic acid) |
| NR1B3 | RARg | Retinoids (e.g. retinoic acid) |
| NR1C1 | PPARa | Fatty acids, prostaglandins, |
| | | phospholipids |
| NR1C2 | PPARb/d | Fatty acids, prostaglandins, |
| | | phospholipids |
| NR1C3 | PPARg | Fatty acids, prostaglandins, |
| | | phospholipids |
| NR1D1 | Rev-erb a, EAR1 | Unknown |
| NR1D2 | Rev-erb b | Unknown |
| NR1F1 | RORa | Unknown |
| NR1F2 | RORb | Unknown |
| NR1F3 | RORg | Unknown |
| NR1H2 | LXRb | Oxysterol |
| NR1H3 | LXRa | Oxysterol |
| NR1H4 | FXRa (rodents only) | Bile acids |
| NR1H5 | FXRb (rodents only) | Lanosterol |
| NR1I1 | VDR | vitamin D (1,25 dihydroxy vitmain D3) |
| NR1I2 | SXR/PXR | Sterols, bile acids, numerous |
| | | xenobiotics and pharmaceutical drugs |
| NR1I3/4 | hCARa/mCARb | Androstans, numerous xenobiotics |
| | | and pharmaceutical drugs |
| NR2A1 | HNF4a | Fatty acids |
| NR2A2 | HNF4g | Fatty acids |
| NR2B1 | RXRa | Rexinoids (e.g. 9-cis retinoic acid) |
| NR2B2 | RXRb | Rexinoids (e.g. 9-cis retinoic acid) |
| NR2B3 | RXRg | Rexinoids (e.g. 9-cis retinoic acid) |
| NR2C1 | TR2 | Unknown |
| NR2C2 | TR4 | Unknown |
| NR2E1 | TLL, TLX | Unknown |
| NR2E3 | PNR | Unknown |
| NR2F1 | COUP-TFI (EAR2) | Unknown |
| NR2F2 | COUP-TFII (ARP1) | Unknown |
| NR2F3 | COUP-TF | Unknown |
| NR2F6 | EAR2 | Unknown |
| NR3A1 | ERa | Estrogens |
| NR3A2 | ERb | Estrogens |
| NR3B1 | ERRa | Unknown |
| NR3B2 | ERRb | Unknown |
| NR3B3 | ERRg | Unknown |
| NR3C1 | GR | Glucorticoids |
| NR3C2 | MR | Mineralocorticoids |
| NR3C3 | PR | Progestins |
| NR3C4 | AR | Androgens |
| NR4A1 | NGFIB, NUR77 | Unknown |
| NR4A2 | NURR1 | Unknown |
| NR4A3 | NOR1 | Unknown |
| NR5A1 | SF-1 | Phospholipids |
| NR5A2 | LRH1 | Phospholipids |
| NR6A1 | GCNF1 | Unknown |

The efficacy of the novel assay was confirmed using nandrolone (6), a commercially available anabolic steroid that is banned by the World Anti-Doping Agency and by professional and collegiate sports. Human serum containing physiologic levels of testosterone (“T”) and dihydrotestosterone (“DHT”) was incubated with agarose-bound AR or control beads. Detection of the steroids can be performed via any effective means in addition to GC/MS, such as liquid chromatography multistage mass spectrometry (“LC/MS/MS”) or any of the other means referenced above. The beads were collected, washed and AR-bound ligands were eluted and analyzed by GC/MS. Test samples include any biological sample including blood, serum, plasma, urine, cerebrospinal fluid, saliva, or other samples that may contain the ligand of interest.

Peaks corresponding to DHT (11.37 min) and T (11.67 min) were detected when using AR, but not when using control beads (FIG. 1A) or beads linked to estrogen receptor α. In a separate experiment, human serum with T and DHT was spiked with 1 nM nandrolone to mimic a “tainted” serum sample. When this “tainted sample was tested an additional AR-specific peak (FIG. 1B, 11.42 min) was identified that does not correspond to the endogenous androgens seen in normal serum (FIG. 1A). Since this material binds AR but does not correspond to a natural androgen, it represents an exogenous androgen. The present screen is useful for detecting the presence of a ligand, like an illicit steroid, without knowledge of its chemical structure. Instead, a ligand is identified by the ability to bind to a known receptor. In this case, the illicit steroid is identified by virtue of its ability to bind AR. This assay has implications for emerging anti-doping legislation and for international testing guidelines and well as for other situations in which a subject needs to be tested for the presence of one or more exogenous ligands.

Materials and Methods

Normal human female serum (2.5 ml) (Gemini Bio-Products, Woodland, CA, cat#100-110F, Lot# H00903Y) was adjusted to contain concentrations of testosterone (T, 15 nM) and dihydrotestosterone (DHT, 3 nM) that are present in normal adult men (7). One portion of the serum was spiked with 1 nM nandrolone. This steroid-containing serum was extracted twice with 2 volumes of ethyl acetate. The two ethyl acetate fractions were pooled, washed twice with water and then dried under nitrogen.

The steroid-screening assay was performed by incubating the ethyl acetate fraction with agarose-bound androgen receptor ligand binding domain (AR). To do so, thioredoxin-linked AR (˜30 μg, Invitrogen) was incubated with 40 μl Ni-NTA agarose (50% slurry) in a total volume of 300 μl of buffer A (20 mM Tris, pH 8, 500 mM NaCl, 5 mM imidazole) at 4° C. for one hour. Ni-NTA beads were pelleted and washed once with 1 ml of ligand binding buffer (30 mM Tris pH 8.0, 150 mM KCl) then resuspended in 300 μl of ligand binding buffer containing 0.4 mg/ml ovalbumin. Immediately prior to assay, the dried ethyl acetate serum fraction was dissolved in 2 ml of ligand binding buffer by incubating for 30 minutes at room temperature and then incubated with the immobilized AR beads for one hour at room temperature followed by one hour at 4° C. Immobilized AR was then pelleted and washed once with 2 ml ligand binding buffer to remove any unbound ligand. To dissociate the bound ligands from receptor, immobilized AR was incubated for 30 minutes at 65° C. in 400 μl PBS and the released ligands were collected in the supernatant. This elution was repeated and the two PBS fractions were pooled, filtered through a 0.22 μm PTFE filter, and extracted twice with three volumes of ethyl acetate. The organic phases were pooled and dried under nitrogen.

The dried samples were derivatized with 20 μl of N,O-bis(trimethylsilyl)trifluoroacetamide containing 1% Trimethylchlorosilane (Pierce) at 55° C. for six hours, then 4 μl of each sample were analyzed by GC/MS run in scanning mode over a mass-to-charge (“M/Z”) range of 50-700 using a ThermoFinnigan Trace DSQ GC/MS system. At the start of each run, the column temperature was held at 50° C. for 1 minute, increased using a gradient of 25° C./min up to 300° C., and held at 300° C. for an additional 8 minutes. A Phenomenex ZB-5 (5% phenyl-95% dimethyl-polysiloxane) column (15 meters, 0.25 mm ID, film thickness 0.50 μm) was used for separation. Following data acquisition, total ion spectra found in the samples were compared to known spectra contained in the NIST 98 Library using the Finnigan Xcalibur software.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

### REFERENCES CITED

1. U.S. House of Representatives, Committee on Government Reform, Bill Comparison: The Clean Sports Act (H.R. 2565) and the Drug-Free Sports Act (H.R. 1862), May 26, 2005 (www.democrats.reform.house.gov/Documents/20050526103452-64714.pdf).
2. D. J. Handelsman, Sci STKE 2004, pe41 (2004).
3. M. H. Sekera et al., Rapid Commun Mass Spectrom 19, 781 (2005).
4. D. H. Catlin et al., Rapid Commun Mass Spectrom 18, 1245 (2004).
5. R. C. Buijsman, P. H. Hermkens, R. D. van Rijn, H. T. Stock, N. M. Teerhuis, Curr Med Chem 12, 1017 (2005).
6. P. M. Holterhus, S. Piefke, O. Hiort, J Steroid Biochem Mol Biol 82, 269 (2002).
7. J. E. Griffen, J. D. Wilson, in Harrison's Principles of Internal Medicine A. S. Fauci et al., Eds. (McGraw-Hill, New York, 1998) pp. 2089.

1. A method of screening for the presence of an exogenous ligand in a subject comprising:
a) obtaining a biological sample from the subject;
b) incubating the sample with a receptor for the exogenous ligand;
c) removing ligand bound to the receptor;
d) analyzing the ligand from step c), wherein
the analysis determines the presence of an endogenous ligand and/or the exogenous ligand.

2. The method of claim 1, wherein determining the presence of the exogenous ligand comprises comparing analytical results from the sample to the results of a sample free of the exogenous ligand.

3. The method of claim 1, wherein determining the presence of the exogenous ligand comprises comparing analytical results from the sample to the results of the endogenous ligand and/or the same or a different exogenous ligand.

4. The method of claim 1, wherein analysis of the ligand comprises the use of any of the following methods, alone or in combination: gas chromatography/mass spectrometry, liquid chromatography/mass spectrometry, liquid chromatography multistage mass spectrometry or nuclear magnetic resonance.

5. The method of claim 1, wherein the ligand is a steroid.

6. The method of claim 5, wherein the steroid is an anabolic steroid and the receptor is an androgen receptor.

7. The method of claim 6, wherein analysis of the bound ligand is conducted using gas chromatography/mass spectrometry and wherein testosterone and dihydrotesterone peaks are compared to peaks of an exogenous steroid which binds to the nuclear androgen receptor.

8. The method of claim 6, wherein the subject is a human or non-human animal and the anabolic steroid is an illicit performance-enhancing steroid.

9. The method of claim 6, wherein the nuclear androgen receptor is attached to a support.

10. The method of claim 9, wherein the support is selected from a bead, nylon membrane, matrix, microtitre well, column, nitrocellulose, agarose, sepharose, polyvinylchloride, polystyrene, diazotized paper, or plastic.

11. The method of claim 1, wherein the biological sample from the subject is blood, serum, plasma, urine, feces, bile, tears, lymphatic fluid, cerebrospinal fluid, or saliva.

12. A screen for determining the presence of an exogenous ligand in a subject comprising the steps of:
a) obtaining a biological sample from the subject;
b) incubating the sample with a receptor for the exogenous ligand;
c) removing ligand bound to the receptor;
d) analyzing the ligand from step c), wherein
the analysis determines the presence of an endogenous ligand and/or the exogenous ligand.

13. The screen of claim 12, wherein determining the presence of the exogenous ligand comprises comparing analytical results from the sample to the results of a sample free of the exogenous ligand.

14. The screen of claim 12, wherein determining the presence of the exogenous ligand comprises comparing analytical results from the sample to the results of the endogenous ligand and/or the same or a different exogenous ligand.

15. The screen of claim 12, wherein analysis of the ligand comprises the use of any of the following methods, alone or in combination: gas chromatography/mass spectrometry, liquid chromatography/mass spectrometry, liquid chromatography multistage mass spectrometry or nuclear magnetic resonance.

16. The screen of claim 12, wherein the ligand is a steroid.

17. The screen of claim 16, wherein the steroid is an anabolic steroid and the receptor is an androgen receptor.

18. The screen of claim 17, wherein analysis of the bound ligand is conducted using gas chromatography/mass spectrometry and wherein testosterone and dihydrotesterone peaks are compared to peaks of an exogenous steroid which binds to the nuclear androgen receptor.

19. The screen of claim 17, wherein the subject is a human or non-human animal and the anabolic steroid is an illicit performance-enhancing steroid.

20. The screen of claim 17, wherein the nuclear androgen receptor is attached to a support.

21. The screen of claim 20, wherein the support is selected from a bead, nylon membrane, matrix, microtitre well, column, nitrocellulose, agarose, sepharose, polyvinylchloride, polystyrene, diazotized paper, or plastic.

22. The screen of claim 12, wherein the biological sample from the subject is blood, serum, plasma, urine, feces, bile, tears, lymphatic fluid, cerebrospinal fluid, or saliva.
